(19) United States
(12) Patent Application Publication
(10) Pub. No.: US 2026/0368945 A1
(43) Pub. Date: Nov. 19, 2026

(54) DEEP LEARNING ASSISTED CONVERSION METHODS OF MICROWAVE SCATTERING SIGNAL TO MEDICAL IMAGING MODALITIES FOR DIAGNOSTIC IMAGING

(71) Applicants: Phawis Thammasorn, Milpitas, CA (US); Lingxiao Wang, San Jose, CA (US); Shih Mo, Milpitas, CA (US)

(72) Inventors: Phawis Thammasorn, Milpitas, CA (US); Lingxiao Wang, San Jose, CA (US); Shih Mo, Milpitas, CA (US)

(73) Assignee: Tiposi, Milpitas, CA (US)

(21) Appl. No.: 19/187,463

(22) Filed: Apr. 23, 2025

(51) Int. Cl. G06T 11/00; A61B 5/0507; G06T 5/10; G06T 5/70

(52) U.S. Cl. CPC G06T 11/00; A61B 5/0507; G06T 5/10; G06T 5/70; G06T 2207/10081; G06T 2207/10088; G06T 2207/10116; G06T 2207/10132; G06T 2207/20064; G06T 2207/20072; G06T 2207/20081; G06T 2207/20084; G06T 2210/41

(57) ABSTRACT

The invention discloses a method and system for deep-learning-assisted conversion of microwave imaging scattering signals into medical imaging modalities. Utilizing a deep learning model and 2D MRI images, the system enables direct mapping of multi-channel microwave imaging scattering signals to organ-specific, high-resolution diagnostic images. This approach offers a cost-effective, non-invasive alternative to traditional imaging techniques, facilitating real-time, modality-specific medical assessments.

### FIELD OF THE INVENTION

The present invention proposes a novel approach for generating high-quality MRI-equivalent images using microwave imaging (MWI) scattering signals. The approach uses a deep learning model to learn a mapping from multi-channel microwave imaging scattering signals to an imaging modality, and output organ-specific image (for example, brain). The training model for the mapping allows direct conversion of microwave signal to the image modalities, effectively synthesizing body-specific and modality-specific scan imagery applicable to diagnostic assessment.

The invention is a part of the effort to create lightweight or portable devices for giving diagnostic assessments that are more accessible to patients outside the large medical center with fully functional imaging machines of required modalities. A major example case is the brain stroke assessment. Determining the occurrence of a stroke typically relies on imaging scans, such as X-ray-based computed tomography (CT) and magnetic resonance imaging (MRI), to identify anomalies in the patient's brain. However, the modality-specific machines may not be available at small medical clinics or centers. The effort is included in our helmet device, which was created to address this problem, and the workflow is summarized below.

### BACKGROUND OF THE INVENTION

Microwave imaging has emerged as a promising non-invasive technique for brain diagnostics, offering a safer and potentially more cost-effective alternative to conventional imaging methods like MRI and CT scans. The effectiveness of microwave imaging in brain diagnostics is highly dependent on the ability of deep learning models to solve inverse scattering problems accurately. These problems involve reconstructing internal structures from observed scattering signals, which is a complex task due to the heterogeneous nature of brain tissues.

A significant challenge in this domain is the generation of comprehensive datasets that reflect the full range of naturally occurring variations in the dielectric properties of human brain tissues under different conditions and frequencies. Existing methods are insufficient in efficiently producing data with the necessary diversity, limiting the accuracy and reliability of deep learning models in solving inverse scattering problems for brain diagnostics.

### SUMMARY OF THE INVENTION

This invention proposes a novel approach for generating high-quality MRI-equivalent images using microwave imaging scattering signals. The approach uses a deep learning model to learn mapping from multi-channel microwave imaging scattering signals to an imaging modality, and output organ-specific image (e.g. brain). The training model for the mapping allows direct conversion of microwave signal to the image modalities, effectively synthesizing body-specific and modality-specific scan imagery applicable to diagnostic assessment.

The invention is a part of the effort to create lightweight or portable devices for giving diagnostic assessments that are more accessible to patients outside large medical centers with fully functional imaging machines of required modalities. A major example practical case is the brain stroke assessment. Determining the occurrence of a stroke typically relies on imaging scans, such as X-ray-based computed tomography (CT) and magnetic resonance imaging (MRI), to identify anomalies in the patient's brain. However, the modality-specific machines may not be available at small medical clinics or centers. The effort is included in our helmet device created to convert scattering signals image for the reconstruction of the region of interest.

### BRIEF DESCRIPTION OF THE DRAWINGS

The accompanying drawing(s), which are incorporated in and constitute a part of this specification, illustrate several aspects described below.

FIG. 1 illustrates a flow chart of a method for generating high-quality MRI-equivalent images using microwave imaging (MWI) scattering signals.

FIG. 2 illustrates system components of the present invention.

FIG. 3 is a schematic description of feature pipeline processing steps from 1D time series signals to wavelet features.

FIG. 4 illustrates the system components of an encoder using Graph Attention Network (GAT) layers and Node features pooling that generate a Z-latent vector used in the Decoder to generate images, and the signal through node features pooling and Z-latent vector from the encoder to the decoder, wherein the encoder contains the node features pooling as well.

FIG. 5. illustrates two imaging examples using the invention. In (a), the left image is generated using the proposed deep leaning system like CNN (Convolutional Neural Network) model, and the right image is generated using MRI (Magnetic Resonance Imaging) for one patient. Similarly, in (b), the left image is generated using the proposed deep leaning system with GAT (Graph Attention Networks) layer, and the right image is generated using MRI (Magnetic Resonance Imaging) for another patient.

### DETAILED DESCRIPTION OF THE PREFERRED EMBODIMENTS

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a compound” includes a plurality of compounds. The following description is provided to enable any person skilled in the art to make and use the invention and is provided in the context of a particular application. Various modifications to the embodiments are possible, and the generic principles defined herein may be applied to these and other embodiments and applications without departing from the spirit and scope of the invention. Thus, the invention is not intended to be limited to the embodiments and applications shown but is to be accorded the widest consistent with the principles, features and teachings disclosed herein.

Principles behind medical images for diagnosis in the present invention can be boiled down to mapping input signal to corresponding matter inside the organ. For microwave imaging (MWI), the signal is the electromagnetic (EM) wave scattered when the wave traverses through non-homogenous organic material (e.g. tissue, white matter, blood, etc.) The scattering signal can be decoded into measurable dielectric properties characterizing known organic matter. For MRI, the signal is the proton alignment affinity toward the input magnetic field decoded into tissues with distinct proton density. For CT, the signal is the attenuation of ionizing X-ray that traverses. Decoding the material-specific attenuation forms the image depicting scanned organs. From these medical imaging mechanisms, modality-specific tissue-specific signal response can be converted into imaging and vice versa. As MWI is the modality offering fastest and non-invasive means to perform acquisition, we utilize this equivalent aspect to convert from microwave imaging signal to the other modalities especially MRI. The process to convert scattering signals image is part of the reconstruction of the region of interest. Although we focus on generating MRI as the main output modality as it is crucial information on brain stroke diagnosis, the algorithm generally applicable to the conversion of some other medical imaging modalities, such as CT.

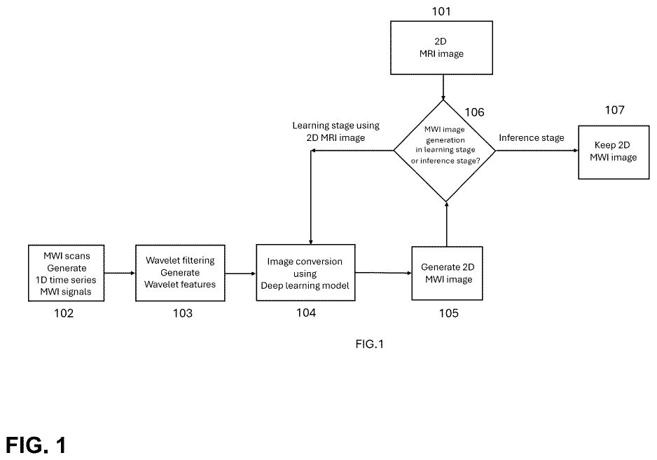

One embodiment is illustrated with reference to FIG. 1, an exemplary flow chart. FIG. 1 is one flow chart embodiment of the proposed disclosure. The embodiment uses 102 for MWI scans generating 1D (one-dimensional) time series MWI signals, 103 for wavelet filtering that generates wavelet features, 104 for image conversion using deep learning model, and 105 for generating 2D (two-dimensional) MWI image. 106 compares the 2D MRI image of 101 with the 2D MWI image in learning stage or just keeps 2D MWI image in inference stage. If the 2D MWI image matches with the 2D MRI image based on some image quality criteria, 107 of the exemplary flow chart is to keep the 2D MWI image. If the 2D MWI image does not match with the 2D MRI image due to not meeting the quality criteria, 104 needs to re-adjust the deep learning model for conversion to generate a better 2D MWI image for comparison with the 2D MRI image, until the new 2D MWI image matches the target 2D MRI image.

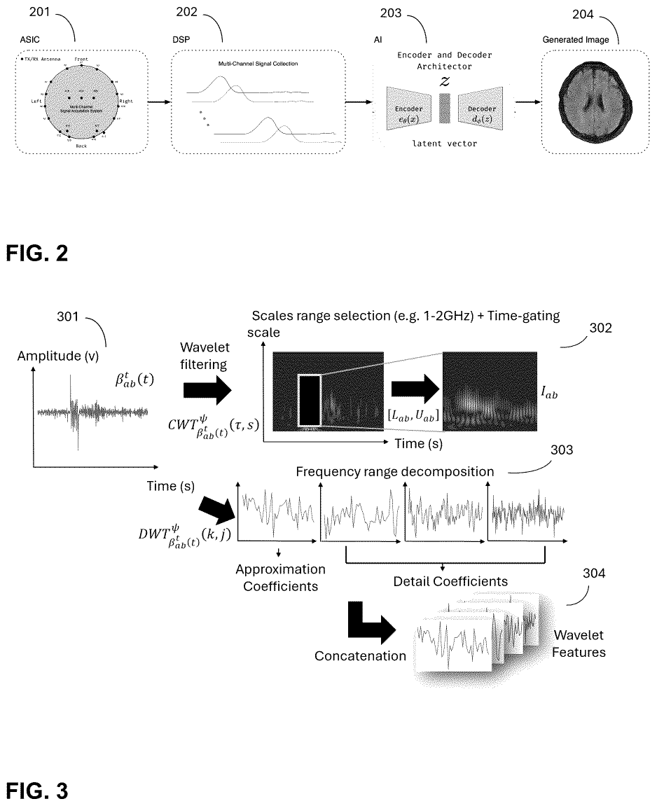

The flowchart of functions 102, 103, 104, and 105 in the embodiment illustrated in FIG. 1 can be mapped to the system components shown in FIG. 2. The proposed system comprises three major components: an application-specific integrated circuit (ASIC), a digital signal processing (DSP) module, and an artificial intelligence (AI) module, which ultimately generates the output image. Function 102 is implemented in ASIC, enabling multi-frequency signal acquisition through a network of software-defined radio transceiver antennas. Function 103, assigned to the DSP module, handles multi-channel signal collection and preprocessing. Function 104, within the AI module, performs image conversion using a deep learning model with an encoder-decoder architecture. The AI system further processes these multi-frequency signals to generate visualizations of the scanned body part and assess potential medical conditions, such as stroke occurrence and affected regions. While the overall system integrates both hardware and software, this patent specifically focuses on the AI component, particularly the DSP-based image formation methodology utilizing a deep neural network.

One embodiment of the ASIC (Application-Specific Integrated Circuit) component 201 in FIG. 2 implements a sequential transmission methodology in which each antenna element (A1-A20) is individually and sequentially designated as a transmitting element while the remaining antenna elements operate in receiving mode. During each transmission cycle, when a specific antenna element (e.g., A1) is activated as a transmitter, the remaining antenna elements (A2-A20) function as receivers, creating multiple simultaneous signal reception pathways. This architecture generates parallel data streams from each receiving antenna, enabling the system to capture comprehensive spatial and temporal characteristics of the propagated signals for enhanced analysis. By structuring the system to maximize the utilization of the antenna array, each element contributes to both transmission and reception phases. While the current embodiment describes a sequential signal acquisition approach—processing signals one channel at a time—the underlying hardware architecture supports parallel acquisition, allowing for future extensions to concurrent data collection across multiple channels. The later sections detail signal acquisition as a sequential process for clarity, but the system remains inherently capable of parallelized operation.

The digital signal processing (DSP) component 202 in FIG. 2 begins with signal acquisition through the ASIC and antenna settings like the embodiment described above. This device positions transceiver antennas around the subject's head. The scanning procedure is initiated by selecting pairs of antennas for transmission and reception, with each unique pair forming a distinct channel. The system repeatedly transmits and records signals across these channels, averaging them to create a single time-domain signal per channel. The averaging is done to remove noise and ensure the stability of the acquired signal. These averaged signals undergo DSP wavelet filtering to generate 2D scalograms representing frequency-time responses. Each channel's scalogram is then frequency-limited to specific ranges (for example, 1-2 GHZ), producing gated and limited responses across all channels that form a consolidated 2D signal for further processing. Finally, a trained neural network model decodes these 2D signal representations to convert them into the target imaging modality.

An AI model in 203 in FIG. 2 takes microwave signal as the input and generates images 204 that resemble modality-specific medical imagery from the signal. The objective of our AI model in FIG. 2 is to perform mapping of MWI signal to imagery from MRI or other modalities. The conversion process can be separated into three major parts, namely signal acquisition, model training, and inference.

In the first part, signal acquisition, the signal is captured by transmitting a signal through an antenna. Then, the signal propagates through the part of the body and is received at another antenna. In one of our implemented devices, we place transceiver antennas surrounding the proximity of the subject's head snuggly placed in our helmet prototype. The scans process by the following steps. Notice that the descriptions here are for signal channel acquisition which can be done both sequentially and parallel with the other channels.
1—At each time, the transceiver system selects two antenna pairs for transmission and receiving respectively. Each antenna pair is referred to as a unique channel. Then the signal is transmitted repeatedly and recorded at the receiver.
2—The signals from all the pairs are average across all to create single time-domain signal representing response from each channel. The averaging is done to remove noise and ensure stability of acquired signal.
3—The average signal undergoes wavelet transform to create 2D scalogram reflecting frequency-time responses.
4—Each channel scalogram is then time-gated and frequency scale and limited to specific corresponding range (e.g., 1-2 GHz). The gated limited responses across all channel form 2D images to be used as input into the deep learning model.

TABLE 1
The summary of symbols, variables, and functions used to describe our
signal processing.

| Variable | Represented meaning |
|---|---|
| x or X | An arbitrary variable representing an |
| | input or signal for demonstrating utility |
| | or some other functions. (e.g. a time |
| | domain signal X(t), or an encoder |
| | network e_{θ} (x)) |
| f | A real-value frequency in frequency |
| | domain. |
| t | A real-value time point in a recorded |
| | time domain signal. |
| τ | An amount of time shift applied to a |
| | function (e.g. mother wavelet function) |
| s | A real-value scaling applied to a function |
| | (e.g. ., mother wavelet function) |
| n | The total number of transceiver antennas |
| | in our system can be designated for |
| | either a transmitter or a receiver at a |
| | time. |
| z | A variable representing feature vector or |
| | latent variable derived as an output |
| | feature from a deep learning network |
| | (e.g. feature from encoder network |
| | z = e_{θ} (x) where z is the derived feature |
| | representing the input x after digested by |
| | the deep encoder network) |
| β_{ab} or β_{ab} (f) | The received signal transmitted from |
| | antenna a and received using antenna b. |
| | β_{ab} is the signal in frequency domain |
| | and have complex value. The variable |
| | is a function of a frequency f. |
| ${β}_{a b}^{T} or {β}_{a b}^{T} ( t )$ | The transformed version of β_{ab} into time $domian . {β}_{a b}^{T} is the signal in time domain$ and have real value. The variable is a function of time t. |
| I_{ab} | $Wavelet features acquired from {β}_{a b}^{T} using$ |
| | wavelet filtering and time gating. |
| I | A set representing the gathered from all |
| | wavelet feature across all available |
| | channel such that |
| | I = [I₁₁, I₁₂, . . . , Iₙₙ] |
| ψ or ψ_{τ,s} | A function represents a unique mother |
| | wavelet used in wavelet transform/ |
| | filtering process. The symbol is a |
| | function of scales and time shift τ. |
| ${CWT}_{{β}_{a b}^{T} ( t )}^{ψ} ( τ , s ) or {CWT}_{{β}_{a b}^{T}}^{ψ} ( τ , s )$ | Continuous wavelet filtering applied to ${β}_{a b}^{T} using ψ wavelet family .$ |
| ${DWT}_{{β}_{a b}^{T} ( t )}^{ψ} ( τ , s ) or {DWT}_{{β}_{a b}^{T}}^{ψ} ( τ , s )$ | $Discrete wavelet filtering applied to {β}_{a b}^{T}$ using ψ wavelet family. |
| L_{ab} | A real-value time lower bound applied to |
| | a time-domain variable from the |
| | transmitter a and the receiver b. The |
| | applied variable is thresholded such that |
| | ${[ X ( t ) ]}_{{L}_{a b}} = \{ \begin{matrix}0 & for t > {L}_{a b} \\ X ( t ) & otherwise\end{matrix}$ |
| U_{ab} | A real-value time upper bound applied to |
| | a time-domain variable from the |
| | transmitter a and the receiver b. The |
| | applied variable is thresholded such that |
| | ${[ X ( t ) ]}^{{U}_{a b}} = \{ \begin{matrix}0 & for t > {L}_{a b} \\ X ( t ) & otherwise\end{matrix}$ |
| ${[ X ( t ) ]}_{{L}_{a b}}^{{U}_{a b}}$ | An operation of applying both U_{ab} and L_{ab} thresholds to the time- domain variable X(t). |
| θ | A variable representing parameters for |
| | neural network. This is used to describe |
| | which associated variable is part of a |
| | deep learning model construct (e.g. the |
| | encoder and the decoder networks) |
| e_{θ}(x) | A function represents an encoder |
| | network. The network take input x and |
| | creates feature representation z. |
| d_{θ}(z) | A function represents a decoder network. |
| | The function take input z to generate 2D |
| | image data from latent variable. |

The following are the expanded technical details of the 4-step scans. Table 1 summarizes the variables we used to describe the signal processing. The pair selection in step 1 defines each channel which denoted by (a, b) where a is the unique id number of the transmitting antenna, and b is the unique id number of the receiving antenna. Let β_{ab} be the signal acquired in step 2 from a channel. In literature, the variable β_{ab} is often expressed in frequency domain although the signal can be practically acquired in either time or frequency domain. We unpack such complex signals into different time-frequency components using wavelet filtering. In our implementation, we capture each channel signal β_{ab} in the time domain and then apply wavelet filtering to obtain a two-dimensional signal. Let

$${β}_{ab}^{T} ( t )$$

represent the signal in time domain. We define our target wavelet-filtered signal after step 4 as I_{ab} with the following definition:

$${I}_{ab} = {[ ‖ {CWT}_{{β}_{ab}^{T} ( t )}^{ψ} ( τ , s ) ‖ ]}_{{L}_{ab}}^{{U}_{ab}} , wherein$$

$$CW {T}_{{β}_{ab}^{T} ( t )}^{ψ} ( τ , s ) = \frac{1}{\sqrt{s}} {\int }_{- ∞}^{∞} {β}_{ab}^{t} ( t ) {ψ}_{τ , s}^{*} ( \frac{t - τ}{s} ) dt$$

In these equations, L_{ab} and U_{ab} denote the time-gated lower and upper bounds of the time-domain signal, respectively, such that any signal

$${β}_{ab}^{t} ( t )$$

outside the range [L_{ab}, U_{ab}] is excluded. We define wavelet filtering via the continuous wavelet transform (CWT), where * denote the complex conjugate, s is the wavelet scale values correspond to the frequencies in the limited corresponding range, and Ørs is the mother wavelet used.

This wavelet filtering allows us to organize the individual patient's microwave 2D signal across all the channel as the input I=[I₁₁, I₁₂, . . . , Iₙₙ] as a tensor with dimensions (n²×Nₛ×max_{ab}(U_{ab}−L_{ab}+1)) where Nₛ is the number of wavelet scales used, and max; (U_{ab}−L_{ab}+1) is the largest timeframe gated among all available channels. Notice that I can be viewed as an n²-channel image with height equal to Nₛ and width equal to the largest timeframe max ab (U_{ab}−L_{ab}+1) across all ab antenna channels. Additionally, padding operations are allowed for the timeframe [L_{ab}, U_{ab}] to ensure dimensions match max_{ab}(U_{ab}−L_{ab}+1). FIG. 3 summarizes the processing of I.

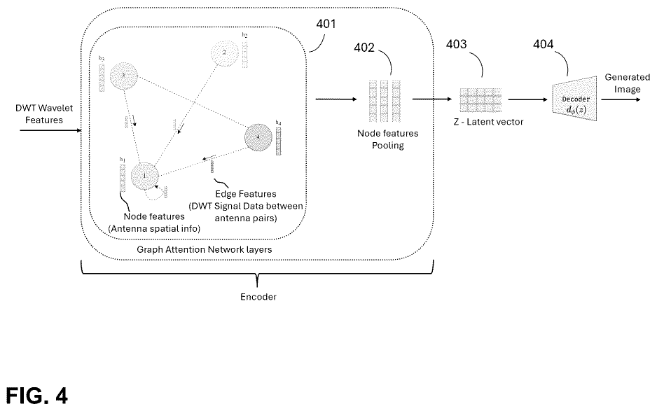

FIG. 3 illustrates a schematic description of feature pipeline processing steps from 1D time-series signals to wavelet features. The antennas with transceivers on the helmet attached to the patient's head can transmit and receive signals. One transceiver transmits pulse signals, and the other transceiver detects signals shown in 301. Such signals are under wavelet filtering to form 2D (2-Dimensional) signals shown in 302 and are used in the Convolutional Neural Network (CNN) model. The signals received shown in 301 can be decomposed into several frequency ranges using Discrete Wavelet Transform (DWT). Such signals are also under wavelet filtering to form 2D (2-Dimensional) signals shown in 303 and are used in the Graph Attention Network (GAT) mode as an alternative method. Like Fourier Transform, we obtain coefficients for different frequency ranges which are called Approximation and Detail Coefficients. These coefficients shown in 303 contain values for different time windows. All the coefficients in 303 are concatenated to form a single 1D (1-Dimensional) vector signal. Such vector signal has wavelet features and is processed by GAT (Graph Attention Network) shown in FIG. 4.

FIG. 4 illustrates the system components of an encoder using Graph Attention Network (GAT) layers, a decoder, and the signal through node features pooling and Z-latent vector from the encoder to the decoder. 401 uses a graph structure where each antenna is represented as a node. The edge weights in this graph are derived from the concatenated DWT (Discrete Wavelet Transform) coefficients, effectively encoding the signal interactions between antenna pairs. Node features are initialized with a dimension of 3, providing a foundation for further feature extraction and learning. The features are initialized with the approximate 3D coordinates of the antenna pads of the microwave imaging device, shown schematically in 201.

401 takes input signal with wavelet features, wherein the node features contain spatial information from the antenna and the node features update their information by learning from connected nodes utilizing the edge features. After forward propagation though the GAT layers in 401, the learned information from the processed signals goes through node features pooling in 402, wherein the learned (updated) information from all the nodes is combined through a pooling operation to become a latent representation, that is, a Z-latent vector, in 403. The Z-latent vector is processed by a decoder in 404 consisting of Residual Blocks and Transpose convolutions. This generates the image in the MRI/CT domain, effectively translating the graph-based representation back into a visual format.

The core of the system in 401 is a sophisticated Graph Attention Network (GAT) comprising multiple layers of Residual Graph Attention Convolutions. Each layer incorporates:
Graph Attention Mechanism: Utilizes both node and edge features to compute attention weights, enabling selective focus on relevant signal interactions.
Residual Connection: Implemented between the input and output of the Aggregation layer, facilitating gradient flow and enabling deeper network architectures.
   Post-convolution processing includes:
Graph Normalization (GraphNorm)
Layer Normalization
ELU (Exponential Linear Unit) activation function

The architecture is further enhanced with skip connections every two layers, improving information flow and gradient propagation. The final graph representation undergoes global mean pooling, followed by a linear projection to the latent space.

The model's architecture is characterized by:
12 GAT layers.
4 attention heads per layer
Hidden dimension of 128.
Latent dimension of 1024 (reshaped to 4×16×16)
   These parameters have been carefully chosen to balance computational efficiency with model expressiveness, enabling the network to capture complex relationships in the input data.

In the second part, for model training of the CNN encoded-based model, the model takes scalograms from all the channels to form a multi-channel image representing a subject. Then, for each subject, an MRI image (or other image modality) is designated as the output such that each wavelet-scalogram image has a real MRI as an expected ground-truth outcome. As depicted below, the mapping is a model using an encoder-decoder neural network where the encoder uses a convolutional neural network (CNN) consisting of convolutional layers to learn a more compact representation of the image and the decoder uses another CNN consisting of deconvolutional layers to restore the representation into an image with the same size as the input. Similarly, for model training of the GAT encoder-based model, the model takes DWT concatenated vectors from all the channels to construct the graph and perform the same training process as defined above for the CNN encoder-based mode. At each training round, the network received input from multiple subjects. Reconstruction loss evaluates the output with the expected MRI outcome, then back propagates the error to adjust the network. After multiple training runs, the network learns the mapping from the microwave wavelet-scalogram image to the MRI modality.

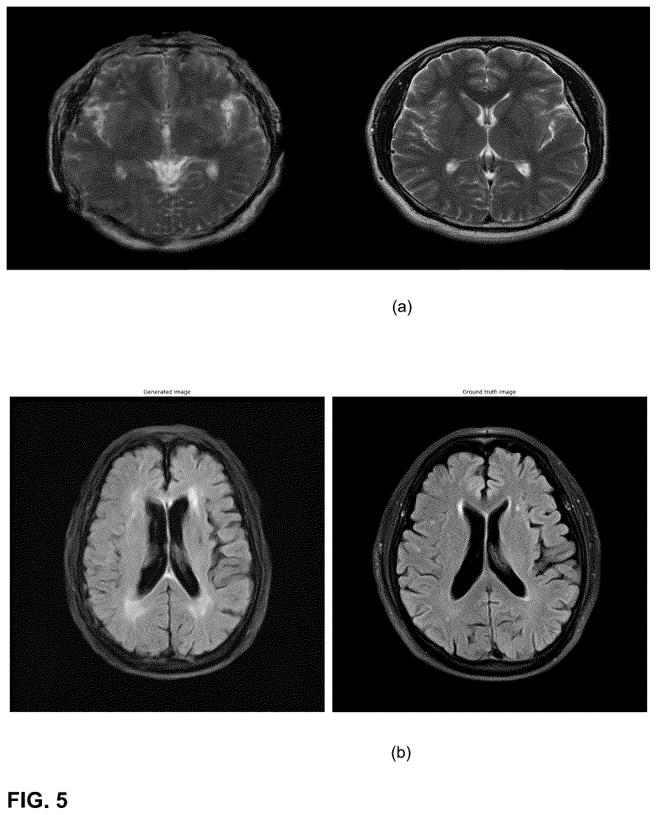

In the third part, inference, the inference run is the process that takes a signal from a new subject and synthesizes a new MRI image. The new subject undergoes the same signal acquisition described above. Then the preprocessed signal is sent to the trained encoder-decoder model to convert into a new MRI-like image. FIG. 5 shows two examples of our system. In FIG. 5 (a), the left-hand side image is the synthesized image after the conversion using Convolutional Neural Network (CNN) model, and the right-hand side image is the ground-truth MRI for one patient. In FIG. 5 (b), the left-hand side image is the synthesized image after the conversion using the GAT (Graph Attention Network) encoder, and the right-hand side image is the ground-truth MRI for another patient.

### I. DEFINITIONS

Unless defined otherwise, all technical and scientific terms used herein have the same meaning as commonly understood by one of ordinary skill in the art to which this invention belongs. As used herein the following terms have the following meanings.

The term “about” when used before a numerical designation, e.g., temperature, time, amount, concentration, and such other, including a range, indicates approximations which may vary by (+) or (−) 10%, 5% or 1%, or any subrange or sub-value there between.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a device or method consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

What is claimed is:

1. A method for converting microwave imaging scattering signals into medical images using deep learning model, comprising:
acquiring multi-channel microwave imaging scattering signals from a target body region;
preprocessing the acquired signals to normalize and remove noise;
inputting the preprocessed signals into a deep learning model trained to map the signals to medical imaging modalities using MRI images;
generating an organ-specific medical image based on the output of the deep learning model.

2. The method of claim 1, wherein the deep learning model comprises:
a plurality of convolutional layers configured to extract spatial and spectral features from the input signals;
a pooling layer configured to reduce the spatial dimensions of the extracted features;
a fully connected layer configured to map the extracted features to a medical imaging space;
a plurality of deconvolutional layers configured to decode spatial information from the extracted features;
an output layer configured to produce the medical image.

3. The method of claim 1, wherein the training dataset comprises paired microwave imaging scattering signals and ground truth medical images.

4. The method of claim 1, wherein the medical imaging modality is selected from the group consisting of MRI-equivalent, CT-equivalent, ultrasound-equivalent, and X-ray-equivalent images.

5. A method for generating MRI-equivalent images from microwave imaging scattering signals using deep learning, comprising:
acquiring multi-channel microwave imaging scattering signals from a subject;
preprocessing the acquired multi-channel microwave imaging scattering signals to normalize and filter noise;
inputting the preprocessed multi-channel microwave imaging scattering signals into a trained deep learning model;
generating an organ-specific MRI-equivalent image based on the model's output.

6. The method of claim 5, wherein the deep learning model comprises:
a convolutional neural network (CNN) configured to extract spatial and spectral features from the input data;
a transformation layer that maps microwave signals to MRI-equivalent images;
an output layer that produces high-resolution organ-specific images.

7. The method of claim 5, wherein the microwave imaging scattering signals are acquired using a multi-frequency, multi-channel transmitter-receiver system.

8. The method of claim 5, wherein the training dataset comprises paired microwave imaging scattering signals and corresponding MRI ground truth images.

9. A system for generating MRI-equivalent images from microwave imaging signals comprising:
a microwave signal acquisition subsystem;
a preprocessing module applying Discrete Wavelet Transform (DWT) to generate DWT coefficients;
a graph construction module for creating a graph structure with edge weights derived by concatenating the DWT coefficients;
a Graph Attention Network model configured to map microwave imaging signals to MRI-equivalent images;
an output module that generates and displays diagnostic images.

10. The system of claim 9, wherein the Graph Attention Network model comprises multiple layers of graph attention mechanisms.

11. The system of claim 9, wherein the Graph Attention Network (GAT) model is trained using supervised learning with a dataset of paired MWI and MRI images.
